(19) United States
(12) Patent Application Publication
(10) Pub. No.: US 2005/0173917 A1
(43) Pub. Date: Aug. 11, 2005

(54) Retractable retention device and device retaining accessory and method for use

(76) Inventors: Eric Kovall, Bedford, MA (US); Andrew F. Paddock, Billerica, MA (US)

Correspondence Address: PERKINS, SMITH & COHEN LLP, ONE BEACON STREET, 30TH FLOOR, BOSTON, MA, 02108, US

(21) Appl. No.: 11/028,000

(22) Filed: Dec. 31, 2004

Related U.S. Application Data
(60) Provisional application No. 60/533,551, filed on Dec. 31, 2003.

(51) Int. Cl. A61M 5/32; A63C 11/00

(52) U.S. Cl. 280/809

(57) ABSTRACT

A retractable retention device for use to stow away an equipment tether or leash that is used to satisfy sports equipment, such as snowboarding and snow skating, safety regulations. The retractable retention device includes means to secure the retraction mechanism and retractable cord to equipment and user, and an internal energy source capable of allowing extension and retraction of the retractable cord. The retractable retention device can be attached to or designed into personal clothing articles, boots, binding, and other sports equipment.

### CROSS REFERENCE TO RELATED APPLICATIONS

This application claims the benefit of U.S. Provisional Application Ser. No. 60/533,551, titled “RETRACTABLE SAFETY LEASH DEVICE AND ATTACHMENT SYSTEM” filed on Dec. 31, 2003, and which is incorporated herein by reference.

### BACKGROUND OF THE INVENTION

This invention relates generally to safety retention devices, and, more particularly, to a retractable sport safety leash device that attaches the equipment to user and retracts when not in use.

For example, in the sport of snowboarding a retention safety leash should attach either the snowboard or one of its bindings to a garment or other equipment fixed or otherwise attached to the user. Hereinafter, the term “accessory” or “user” may be used interchangeably and will refer to boots, socks, pants, jacket, shirt, or any garment that during normal activities associated with the sport is attached to the person practicing the sport. The term “equipment” or “object” may be used interchangeably and will herein refer to snowboard, snowboard bindings, skateboard, snowskate, surfboard, and any other sporting equipment requiring a leash. The term “tether” may refer hereinafter to rope, cord, string, twine, and any other flexible, pliable material having the capability to be coiled.

Most users that practice the sport use conventional safety retention devices to appease the ski resort personnel as the devices intended safety duties are very rarely ever called into practice. Conventional retention devices, such as snowboard leashes, are designed with inherent problems such as lack of convenience or nuisance when not in use either on the equipment to be retained, such as a snowboard or binding, or when fixed to a garment or equipment on the user, such as a pant leg or a boot. The lack of convenience may be illustrated in the loose length of leash or tether that is always present with conventional leashes.

Another problem with conventional retention devices is that they are dangerous when in normal use because any excess retention device length that dangles from the equipment can cause injury to the user in several possible ways. For example, if the device is a snowboard leash attached to a snowboard, the device can get caught under the edges and cause the user to fall. The device can also get snagged on anything ranging from trees to people either while the user is riding on the chair lift or riding down the mountain.

A still further problem arises when transporting the equipment on an exterior automobile ski rack. Conventional devices can flap in the wind, possibly harming people, other equipment, or the automobile.

Therefore, a primary object of the present invention is to provide a convenient retractable retention device that will overcome the shortcomings, previously described, of conventional retention devices and can be readily adapted for use with equipment such as a snowboard or the like.

A second object is to provide a retractable retention device that provides for retracting a leash into a casing that can be of any size or style.

A third object of the present invention is to provide a retractable retention device that provides for a casing that can be firmly and conveniently stowed within an area in the structural sections of an equipment snowboard binding or the like.

A fourth object is to provide a retractable retention device wherein the structural section of the equipment, for example snowboard binding, is the casing of the retraction mechanism.

A fifth object is to provide a retractable retention device that can be inserted into a pouch within a snowboard binding strap.

A sixth object is to provide a retractable retention device that can be laid up within the structural housing of a surfboard.

A seventh object is to provide a retractable retention device that can be housed within a snowskate board.

An eighth object is to provide a retractable retention device that may be easily removed from some of the equipment previously described if desired.

A ninth object is to provide a retractable retention device that allows for normal user movement and normal operation of the equipment.

A tenth object is to provide a retractable retention device that, when engaged, relieves retraction tension on the tether, user, and equipment.

An eleventh object is to provide a retractable retention device that is lightweight and does not affect the overall balance of the equipment.

A twelfth object is to provide a retractable retention device that provides adequate safety strength to prevent runaway equipment.

A thirteenth object is to provide a retractable retention device that may also be used as a theft deterrent lock mechanism whether in function, fashion, or both.

A fourteenth object is to provide a retractable retention device that satisfies recognized safety criteria.

A fifteenth object is to provide a retractable retention device that is economical to manufacture and capable of being readily mass-produced.

A sixteenth object is to provide a retractable retention device that can be fabricated to be housed within a pair of snowboard pants.

A seventeenth object is to provide a retractable retention device that can be internally installed within a snowboard sock.

An eighteenth object is to provide a retractable retention device that can be built into a snowboard boot as an internal feature within the tongue, ankle, heel, or the like.

### SUMMARY OF THE INVENTION

The objects set forth above as well as further and other objects are achieved by the present invention. The solutions and advantages of the present invention are realized by the illustrative embodiment of the present invention described hereinbelow.

The retractable retention device of the present invention includes a stored energy retraction initiating device, a casing that can conceal or hold a retracted cord or leash, a locking device that can hold the cord in an extended or a retracted position, and a release device that can release the cord from its locked state. The stored energy retraction initiation system (either automatic or manual) retracts the cord by means of a stored energy source such as, for example, a coiled copper piece, a spring, or any mechanical device that utilizes “stored energy”. Pulling the retractable cord to the suitable length engages the cord at the given length. The cord can be wrapped around or attached to the retraction/extension device such that when the cord is fully extended an “outward snap” or “release button” releases the cord, and the internal energy or “spring like mechanism” returns the cord to its coiled storage position.

The retractable retention device includes a casing that can be securely (i) attached to an unused binding insert on the equipment binding such as a snow board, (ii) adhered to the topsheet of the equipment, (iii) wrapped around any binding strap, binding highback, the outside of either binding heelcup, or the outside of either binding baseplate, or (iv) affixed within or adhered to the outside of user clothing. The retractable retention device can be mounted in several ways including, but not limited to, an attachment clip, such as a swivel clip, that enables the user to store the retention device conveniently on any conventional pant belt or anywhere on the equipment or binding that does not interfere with ease of function.

For a better understanding of the present invention, together with other and further objects thereof, reference is made to the accompanying drawings and detailed description.

### BRIEF DESCRIPTION OF THE SEVERAL VIEWS OF THE DRAWING

FIG. 1A is a pictorial view of the present invention in use in conjunction with a snowboard;

FIG. 1B is an exploded pictorial view of the several separate parts of the retractable retention device of the present invention;

FIG. 2 is an exploded pictorial view of the housed cord and spring of the retractable retention device of the present invention;

FIGS. 3A and 3B are top and bottom pictorial views, respectively, of the retractable retention device of the present invention when ready for attachment and fully retracted;

FIGS. 4A and 4B are top and side views of a clip used as an attachment mechanism for the retractable retention device of the present invention;

FIG. 5 is a pictorial view of a binding strap used as an attachment mechanism for the retractable retention device of the present invention;

FIG. 6 is a pictorial view of an adhesive coated casing used as an attachment mechanism for the retractable retention device of the present invention;

FIG. 7A is a pictorial view of a built-in pants pouch that can house the retractable retention device of the present invention;

FIG. 7B is a partial view of the clothing attachment system of FIG. 10A;

FIGS. 8A and 8B are side and front views, respectively, of a boot illustrating various locations shown in phantom for locating the present invention of FIG. 1B therein;

FIGS. 9A and 9B are side and front views, respectively, of a boot binding illustrating various locations shown in phantom for locating an integral retraction retention device therein;

FIG. 10A is pictorial view of an alternative attachment means for securing the present invention to a user;

FIG. 10B is a partial view of the alternative attachment means of FIG. 10A;

FIG. 11 is a pictorial view of an alternative embodiment of the present invention shown integral with a snowboard; and

FIG. 12 is a pictorial view of a retraction stop mechanism embodiment.

### DETAILED DESCRIPTION OF THE INVENTION

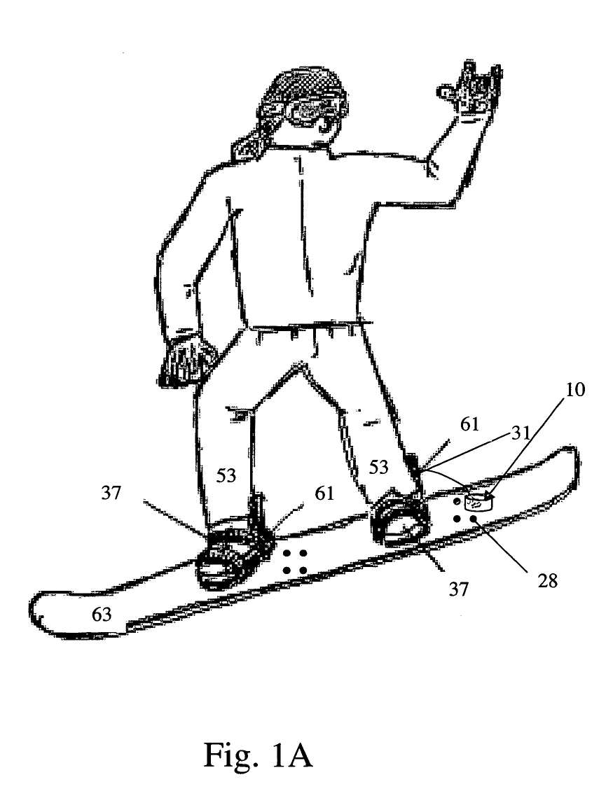

The present invention is now described more fully hereinafter with reference to the accompanying drawings, in which various illustrative embodiments of the present invention are shown. Similar reference numbers in the following drawings denote similar elements throughout the several views of the drawings. FIG. 1A illustrates the present invention of the retractable retention device 10 used in conjunction with a snowboard user outfitted with sports equipment (for example, bindings 61 and snowboard 63) and garments (for example, pants 53 and boots 37).

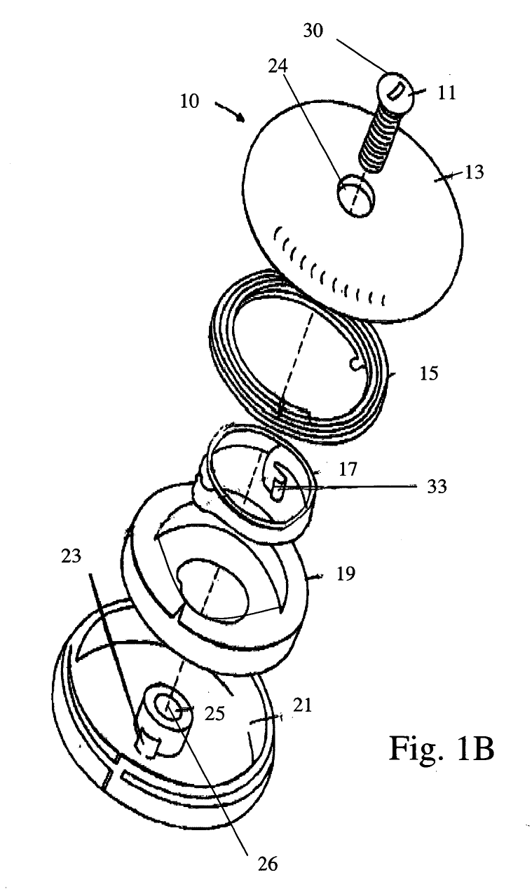

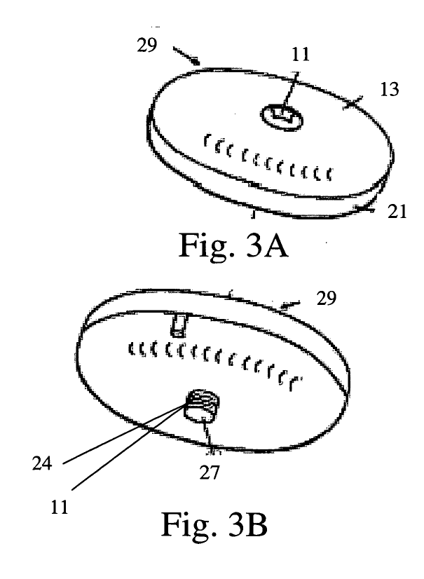

Referring now to FIGS. 1B and 2, the retractable retention device 10 includes screw or bolt 11, top cap 13 of casing 29, cord or tether 15, biasing means 17 (for example, a coil spring or an elastomer), tether race 19, and bottom cap 21 of casing 29. The tether 15 is stored within the race 19 when retracted. The tether 15 may be made of any material that can be drawn into filament geometry, capable of being wound or coiled, and has sufficient tensile strength to withstand the loads under which the device shall be intended to operate. The tether 15 should also be made of a material that is tough to cut, or withstand high shear loads since the present invention may be used as a lock or theft deterrent device. The coil spring 17 may include a fold 33 capable of interconnection with the casing bottom cap 21. Fold 33 can wrap around the hemispherical protrusion 23 in the bottom cap 21 and connects similarly to the race body 19 as folded into a part of the race 19. Also shown is a boss 25 of bottom cap 21 which may be a bearing surface for the race 19 to rotate around as the tether 15 is engaged and retracted, as well as a mating surface hole for the threaded member 11 to be inserted through. Tether 15 may have attachment means 31 (shown illustratively in FIG. 4) fixed to the free end 75 of tether 15 in order to attach the user or equipment to tether 15 when retractable retention device 10 is used for its intended purpose. Top cap 13 and bottom cap 21 may be manufactured as one solid unit or two units to form casing 29 which houses all of the components described above.

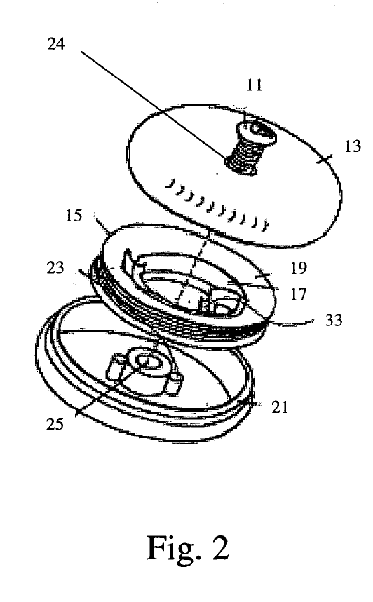

Referring to FIGS. 3A and 3B, bottom cap 21 and top cap 13 are sufficiently sized to fit securely together through engagement of threaded member 11 with mating boss hole 26 (FIG. 1B), the completed assembly forming casing 29. Casing 29 is not limited to a two-part system as described herein. The shape of the casing 29 and thus top cap 13 and bottom cap 21 may be of any shape suitable to the particular application. Top cap 13 and bottom cap 21 may be snapped together if the outer and mating inner edge of the caps is made to form an interference fit or threaded connection with suitable cooperating lips for snapping or screwing to caps together.

Casing 29 can be made of a number of different materials that can be formed to the desired shape and with the necessary tolerances for mating caps 13, 21. A preferred material should have a low thermal expansion coefficient in order to maintain shape and tolerances with the other components of the retractable retention device 10 when exposed to varying temperature changes that are inherent with the sports of, for example, snowboarding, surfing, and snowskating which may be on the order of magnitude of 100 degrees Fahrenheit or more. The casing material is preferably resistant and flexible.

The threaded portion 27 of the screw 11 extends out of bottom cap 21 at predetermined distance and ready to be screwed into any suitably threaded hole 28 within snowboard 63 (FIG. 1A), snowboard binding, snowskate board, skateboard, snowboard boot or the like. The screw 11 may be inserted through casing holes 24 which may be at any location within the casing 29 and may be either threaded holes or non-threaded holes. The head 30 of the bolt 11 may be slightly larger in diameter than casing holes 24 and boss hole 26 so that bolt 11 will constrain the entire retractable device 10 securely to whichever piece of equipment or clothing is chosen.

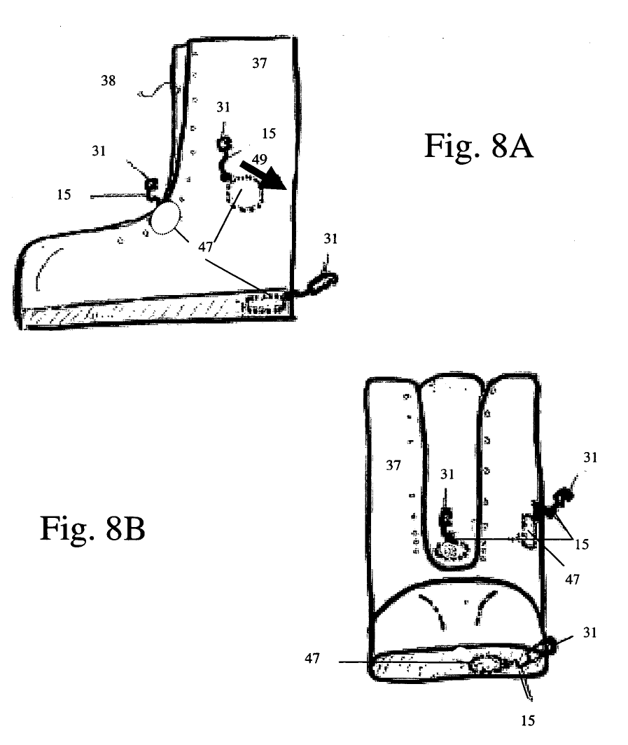

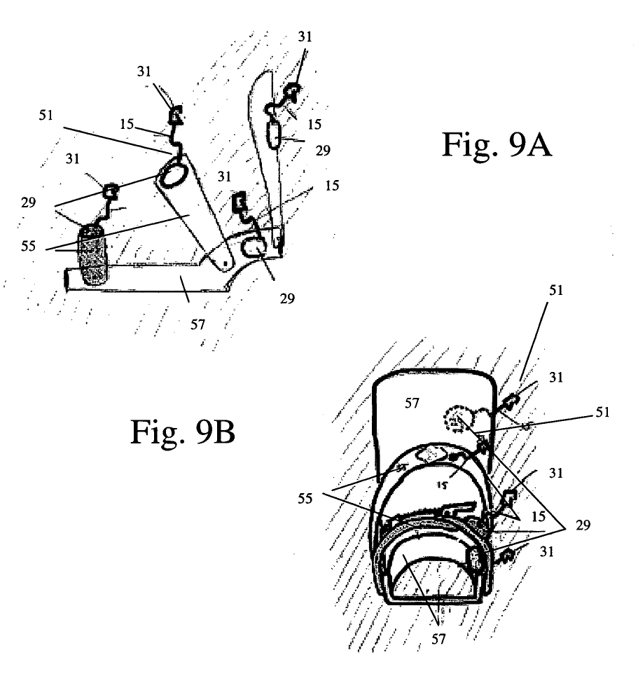

FIGS. 4A, 4B, 5, 6, 10A and 10B further illustrate embodiments to externally secure the device 10 to equipment or user. The attachment means shown in FIGS. 4A and 4B is similar to a money or belt clip style fastener that includes a clip 39 attached to casing 29. Clip 39 is preferably made of a strong material with a high strain to failure and high flexural strength characteristics, but easy to form into the desired complex shape, for example steel, plastics, aluminum alloys, polymers, and metal alloys having similar characteristics. Clip 39 may be of any suitable size for the desired device 10 attachment location on a snowboard binding strap 55 or highback 57 (shown in FIG. 9) or snowboard boot (see FIG. 8).

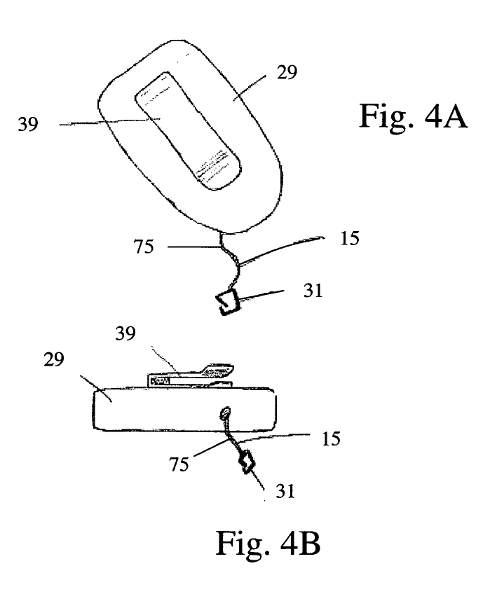

Another embodiment of the present invention includes an attachment mechanism, shown in FIG. 5, having strap 41 which is connected to casing 29 through strap mount 43, which is either fixedly or removably mounted to casing 29 through conventional means. Strap mount 43 may be formed directly into or integral to casing 29 during the molding or forming of casing 29. Strap mount 43 may also be a separate piece that may be secured to the casing 29 through conventional means. Strap 41 can be wrapped around any binding strap, binding highback, heelcup, or baseplate, bootlaces, as well as around the user's clothing. Strap 41 is sized according to its intended mating feature. The strap 41 ends may connect together through a snap button, hook and loop, removable adhesive, or clip mechanism. The strap 41 should be made of a flexible material, fabric or such suitable material for strength and flexibility.

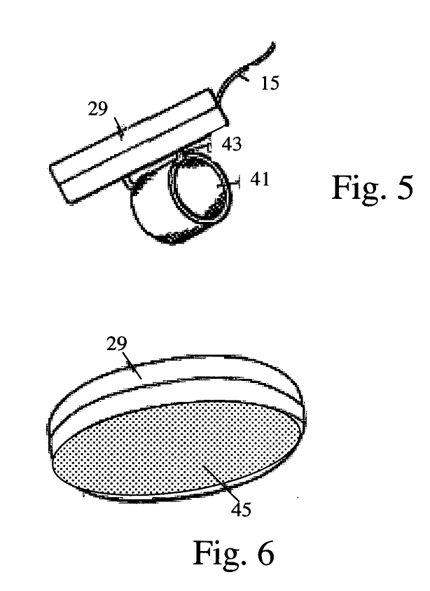

Referring now to FIG. 6, another embodiment of the present invention includes an attachment mechanism having adhesive surface 45 that can be used to attach the casing 29 to any flat or compatibly contoured surface on the equipment or user of the sporting equipment.

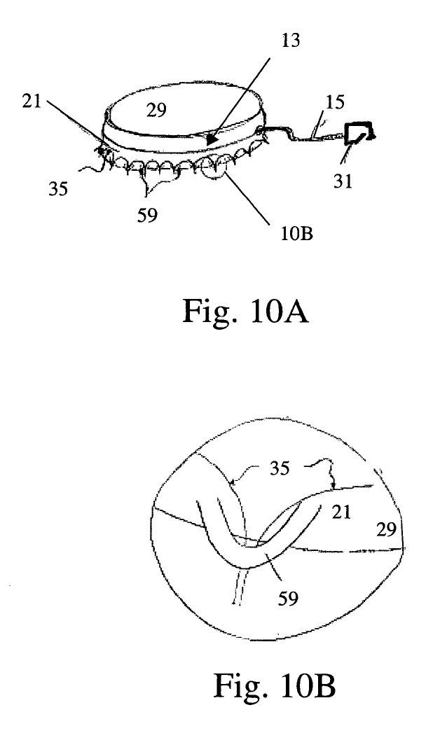

Referring now to FIGS. 10A and 10B, another embodiment of the present invention includes an attachment means having plurality of holes or loops 59 formed along the outer surface of casing 29 through which thread or fiber 35 can be inserted or woven. Fiber 35 can be sewn through the holes or loops and into the user's clothing, boot, binding, or any suitable surface on the sporting equipment, garment 20, boot 37, pant leg 53, binding strap 55, and binding structure 57, thereby weaving or attaching device 10 semi-permanently to the user or user's equipment or apparel. If the mating surface on the equipment is a hard surface, then it must contain appropriate mating holes or loops 59 prefabricated to accept the thread or fiber 35. Casing holes or loops 59 may be either formed during a secondary process during manufacture of the casing 29 or during the primary process of forming the casing 29. Casing holes or loops 59 may be located either circumferentially around the edge of the circular or otherwise shaped casing 29 or at any other suitable location on the casing 29.

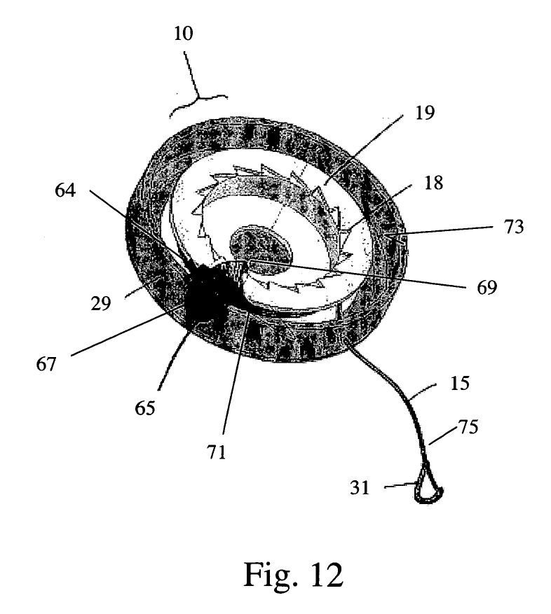

Referring to FIG. 12, another embodiment of the present invention includes a retraction stop mechanism 64 that maintains the extended tether 15 at a certain length desired by the user during use of the retraction retention device 10. The retraction stop mechanism 64 includes a stop release actuator 65 having an actuator head 67, a protrusion 69, and an elongated portion 71. The stop release actuator 65 is made of plastic, thin metal, polymer, graphite, or other sufficiently elastic material such that when actuator 65 is depressed or otherwise deformed it will spring back or return to an initial or locked position without permanent deformation of actuator 65. The elongated portion 71 is attached to the inside surface 73 of the casing 29. The elongated portion 71 can be attached by conventional means including, for example, rivet, pin, press-stud, screw, weld, braze, nut/bolt, and adhesive. The actuator 65 can also be pivotally attached to the casing 29 by use of same or similar conventional attachment means listed above. Protrusion 69 is configured to cooperate with and engage teeth cutouts 18 of race 19. Pressing on or actuating actuator 65 laterally moves protrusion 69 inward and away from teeth cutouts 18 thereby disengaging the protrusion 69 from within the teeth cutouts 18. Alternatively, actuator 65 can be actuated or rotated upward to an angular displacement to vertically raise the protrusion 69 above teeth cutouts 18, thereby disengaging protrusion 69 and teeth cutouts 18. Once protrusion 69 and teeth cutouts 18 are disengaged, the biasing means 17 can recoil thereby retracting extended tether 15 back into casing 29. Actuator head 67 extends outside of the casing 29 and is accessible by the user. The casing 29 has an aperture (not shown) to receive Actuator 65 as it transverses inward and outward or upward and outward to disengage and engage protrusion 69 and teeth cutouts 18.

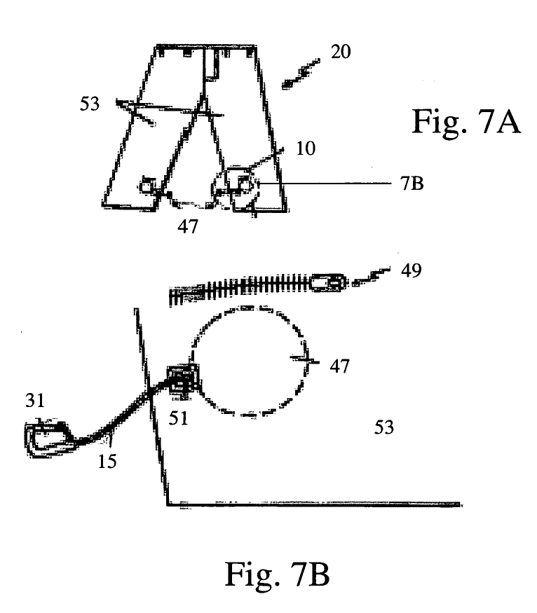

Referring now to FIGS. 7A and 7B, the retractable retention device 10 is housed within a user's article of clothing, for example pant leg 53. The clothing attachment system 20 may include a pouch or pocket 47 that is manufactured within the garment 20. The pocket 47 may have a closing device 49 to keep the retractable retention leash device 10 from falling out of the pocket 47. The closing device 49 may comprise a zipper, button, snap, hook and loop, or resealable adhesive. The pouch 47 also has a small tether hole 51 where the tether 15 protrudes from the internal cavity of the pouch 47 in the pants 53 to the outside where the user may access the tether attachment means 31. The pocket 47 must be suitably larger than the casing 29 of the device 10. The tether hole 51 is preferably larger in diameter than the tether 15 but smaller than that of the leash clip 31 so that upon retraction, the free end 75 of tether 15 and the clip 31 do not retract back into the inside of the pants 53 within the pocket 47.

An alternative internal garment housing system may not have a pocket closing device 49. Device 10 may be installed within the garment 20 at the time of manufacture. This option will be common to all of the following embodiments regarding internally housing the device 10 within any of the equipment or accessories pertinent to the sports of snowboarding, skateboarding, surfing, and snowskating.

Referring to FIGS. 8A and 8B, the retractable retention device 10 may be internally stored within or integral with, for example a snowboard boot 37. Device 10 can be positioned in any location within the boot 37 because the size of the retractable retention device 10 is small enough to fit anywhere in the shell or bladder 42, tongue 38, or sole 40 of a boot 37. FIGS. 8A and 8B illustrate three possible locations for a pocket 47 housing the retractable retention device 10, one of which illustrates a pocket closing device 49. Pocket 47 is preferably formed in boot 37 at the time of manufacture. An alternative embodiment of the present invention eliminates casing 29 since boot 37 acts like a casing when the biasing means 17, tether 15, and other internal components of the device are positioned in a mold that will form a cavity around the internal components during the manufacturing process.

Yet another embodiment of device 10 includes an attachment means within the snowboard binding 61 (FIGS. 9A and 9B). Four locations within the binding 61 have been shown for illustration purposes only and are not intended to limit the present invention to the embodiments disclosed. Two locations are within binding structure 57 and two locations are within each of straps 55. Snowboard binding straps 55 are often made of flexible fabric material within which a retractable leash device casing 29 can be stored. The structural part of the binding 57 may be injection or stamp molded plastic or composite material. Device 10 may be manufactured integral to the structure 57 during binding fabrication having a tether hole 51 for the tether 15 to come through to the outside surface and thus accessible to the user.

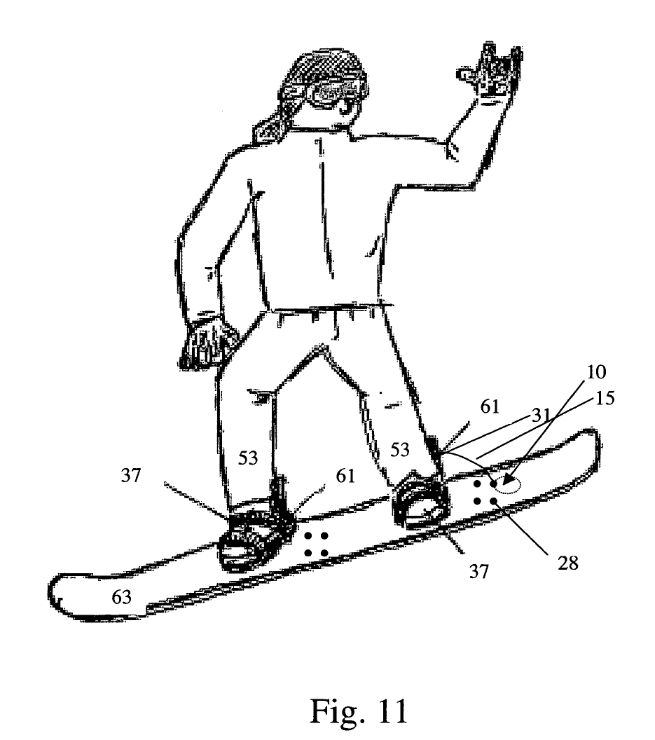

Referring to FIG. 11, retractable retention device 10 (shown in phantom) may be manufactured integral within the equipment, for example snowboard 63, surfboard, or snowskate boards. A fabrication process for this equipment may eliminate the need for casing 29, as disclosed above.

To use the retractable retention leash device 10, the user first attaches casing 29 securely by any suitable attachment means described above to either the user or the equipment, such as binding 61 and snowboard 63. User then grasps the leash attachment means 31 of the free end 75 of tether 15 and secures it to user or to equipment with attachment means 31. Tether 15 can be locked in extended position by stop mechanism 64 (FIG. 12). To retract tether 15 back into its storage position within casing 29, the actuator 65 is actuated to allow the internal energy biasing mechanism 17 to retract the tether 15 into the casing 29.

It is to be fully understood that the invention is not limited in its application to the specific details of construction and to the arrangements of the components set forth in the previous description or illustrations. The invention is capable of other embodiments and of being practiced and carried out in various ways. Also, it is to be understood that the phraseology and terminology employed herein are for the purpose of the description and should not be regarded and limiting. It is to be understood that the invention is not limited in its application to the details of construction and to the arrangements of the components set forth in the previous description or illustrated in the drawings. The invention is capable of other embodiments and of being practiced and carried out in various ways. Also, it is to be understood that the phraseology and terminology employed herein are for the purpose of the description and should not be regarded as limiting. It should be realized this invention is also capable of a wide variety of further and other embodiments within the spirit and scope of the appended claims.

1. A retractable retention device to tether sports equipment to a user, said device comprising:
a tether line of a predetermined length having an first end and a second end, said second end having an attachment means for securing said tether line to the sports equipment or the user;
a first cover having a first cavity and a first edge circumscribing said first cavity;
a second cover having a second cavity and a second edge circumscribing said second cavity, said second cover further having a protrusion extending outward from said cavity;
tether line retraction means attached to said first end, said tether line retraction means includes a biasing means having a recoil extension sized to cooperation with said second cover protrusion;
a race having an outer edge sized to be received within said cavity of said second cover, said race having a recess sized to receive an outer edge of said tether line retraction means, said race having a protrusion extending outward from said recess capable of cooperating with said recoil extension, said race having a connector capable of cooperating with said protrusion of said second cover,
said first and second covers forming a casing when said edges are joined together;
said casing having a chamber formed by the mating of said cavities, said casing having an hole from which said tether line extends therefrom and retracts therein and which restricts said attachment means from entering said casing;
said chamber being of sufficient size to encase said tether line and said tether line retraction means; and
means for attaching said casing to the sports equipment or the user.

2. The device according to claim 1 wherein said means for casing attachment comprises:
a threaded member having threaded portion of predetermined length greater than a thickness of said casing and a head at one end of said threaded portion;
said first and second covers each further including a hole sized to receive said threaded portion and to restrict said head from entry into said holes;
whereby said threaded member extends through and beyond said casing and is capable of being threaded into a threaded hole of the sports equipment or an object attached to the user.

3. The device of claim 1 wherein said casing attachment means comprises an adhesive device attached to an outer surface of said casing for adhering said device to the sports equipment or the user.

4. The device of claim 1 wherein said casing attachment means comprises:
a mount connected to a side of said casing; and
said mount includes an opening capable of receiving a strap.

5. The device of claim 1 wherein said tether line retraction means further comprising a locking mechanism.

6. The device of claim 5 wherein said locking mechanism comprises:
an actuator attached to said casing extending outward therefrom, said actuator having a protrusion extending radially inward within said casing;
said race further comprising a plurality of teeth cutouts to engage said protrusion,
whereby said tether line retractions means is restrained when said protrusion and said plurality of teeth cutouts are engaged.

7. The device of claim 1 wherein said means for casing attachment is integral to the sports equipment.

8. The device of claim 7 wherein the equipment is a snowboard.

9. The device of claim 7 wherein the equipment is a binding.

10. The device of claim 7 wherein the equipment is a skateboard

11. The device of claim 7 wherein the equipment is a snowskate.

12. The device of claim 1 wherein said means for casing attachment is integral to a garment of the user.

13. The device of claim 1 wherein said securing attachment means comprises a plurality of holes along an outer surface of said casing, whereby said device is attached to the equipment or user by threading a fiber through said plurality of holes and into the equipment or clothing of the user.

14. A garment, such as a boot, pair of pants, a sock, a shirt or a jacket, adapted to store a retractable retention device during use of same comprising:
a pouch associated with the garment having an opening sized to receive said retractable retention device and a hole sized to receive a tether line of said device and to restrict an attachment device connected to one end of said tether line; and
a closing device to close said opening after said device is placed within said pouch.

15. The garment of claim 12 wherein said closing device is a zipper.

16. The garment of claim 12 wherein said closing device is a button.

17. The garment of claim 12 wherein said closing device are hook and loop.

18. The garment of claim 12 wherein said closing device is resealable adhesive.

19. A sports equipment, such as a snowboard, snowboard bindings, skateboard, snowskate or surfboard, comprising:
an integral retractable rentention device;
said retracted retention device including:
a tether line of a predetermined length having an first end and a second end, said second end having an attachment device for securing said tether line to the user;
a tether line retraction device attached to said first end, said retractable retention device including a biasing means having a recoil extension;
said sports equipment having a cavity sized to contain said tether line and said tether line retraction device, a protrusion within said cavity sized to cooperate with said recoil extension, and said sports equipment having a hole sized larger than said tether line and smaller than said attachment device.

20. The sports equipment of claim 19 further comprising a race having an outer edge and a depth sized to be received within said cavity, said race having a recess sized to receive an outer edge of said tether line retraction device, said race further having a protrusion extending outward from said recess capable of cooperating with said recoil extension, said race further having a connector capable of cooperating with said protrusion of said cavity.
